(19) United States
(12) Patent Application Publication
(10) Pub. No.: US 2013/0175477 A1
(43) Pub. Date: Jul. 11, 2013

(54) CORROSION INHIBITOR FOR HIGH TEMPERATURE ENVIRONMENTS

(71) Applicants: Olusegun Matthew Falana, San Antonio, TX (US); Khalid Ali Shah, Calgary (CA); Thomas P. Wilson, JR., Floresville, TX (US); Frank Zamora, Schwartz, TX (US)

(72) Inventors: Olusegun Matthew Falana, San Antonio, TX (US); Khalid Ali Shah, Calgary (CA); Thomas P. Wilson, JR., Floresville, TX (US); Frank Zamora, Schwartz, TX (US)

(73) Assignee: Clearwater International, LLC, Houston, TX (US)

(21) Appl. No.: 13/348,267

(22) Filed: Jan. 11, 2012

(51) Int. Cl. C23F 11/167; C07D 265/30; C07F 9/09

(52) U.S. Cl. 252/389.23; 558/208; 544/106

(57) ABSTRACT

Corrosion inhibiting and management systems and for methods of making and using same are presented, where the corrosion systems comprise a reaction product or mixture of reaction products of medium to high carbon count phosphate ester or mixture of medium to a high carbon count phosphate esters and an amine or mixture of amines and where the reaction product or products form a partial or complete coating on surfaces of pipe, piping, pipeline, flowline, and other downhole equipment in contact with aqueous and non-aqueous fluids to protect the surfaces from corrosion at temperature up to up to 750° F. (398.9° C.), and especially, for use in applications using low to moderate temperature geothermal fluids.

### BACKGROUND OF THE INVENTION

1. Field of the Invention

Embodiments of this invention relate to high temperature corrosion inhibiting and management systems stable to temperature up to 600° F. (315.5° C.) and to methods of making and using same.

More particularly, embodiments of this invention relate to corrosion inhibiting and management systems and for methods of making and using same, where the corrosion systems comprise a reaction product or mixture of reaction products of medium to high carbon count phosphate ester or mixture of medium to a high carbon count phosphate esters and an amine or mixture of amines and where the reaction product or products form a partial or complete coating on surfaces of pipe, piping, pipeline, flowline, and other downhole equipment in contact with aqueous and non-aqueous fluids to protect the surfaces from corrosion at temperature up to up to 750° F. (398.9° C.), and especially, for use in applications using low to moderate temperature geothermal fluids.

2. Description of the Related Art

In prior art, there are a number of methods that have been developed for corrosion management at high temperatures. One method involves the use of non-aqueous systems, which, due to the nature of oil-based fluids, coats metals and protects corrosion of downhole tools offering a reliable corrosion management system. Other methods involves the use of metal oxides, metal carbonates (see U.S. Pat. No. 5,312,585) and metal salts (see U.S. Pat. No. 6,620,341). Other methods involved the use of quaternary salts of polyalkylene polyamines (see U.S. Pat. No. 5,275,744), diethylene triamine condensation with triester of fatty acids (see U.S. Pat. No. 3,653,452) and adehyde-thiol condensate (see U.S. Pat. No. 7,216,710). In the case of metal oxides or salts (e.g., molybdates), not only are the salts cost prohibitive, compatibility with drilling fluids is a limiting factor to their use. Corrosion protection is complicated by degradation of thiols, carbamates, sulfates, etc. resulting in secondary corrosion, especially resulting from the exposure of metals to sulfur dioxide.

Corrosion management under conditions of high temperature is still a problem in drilling for fluids and/or solid minerals. Moreover, corrosion management is now a problem in the burgeoning activities using geothermal energy sources, which has brought corrosion management under extreme or hash conditions to the for front. Thus, there is a need in the art for corrosion management systems that have high thermal stability for use in aqueous and non-aqueous systems.

### SUMMARY OF THE INVENTION

Embodiments of this invention provide corrosion inhibiting and corrosion management systems including a reaction product of a medium to high carbon count phosphate ester or a mixture medium to high carbon count phosphate esters and an amine or a mixture of amines, where the reaction product forms a partial or complete coating on surfaces of piping, flowlines or other downhole equipment in contact with an aqueous or non-aqueous fluid. The medium to high carbon count phosphate esters comprise reaction products between a phosphate donor such as phosphorus pentoxide and medium to high carbon count alcohols, i.e., alcohols having between 6 and 24 carbon atoms. In certain embodiments, the alcohols have between 8 and 20 carbon atoms. In certain embodiments, the alcohols have between 8 and 18 carbon atoms. In certain embodiments, the alcohols have between 8 and 16 carbon atoms. In certain embodiments, the alcohols have between 8 and 14 carbon atoms. In certain embodiments, the alcohols have between 8 and 12 carbon atoms.

Embodiments of this invention provide methods for inhibiting and managing corrosion in high temperature environments, where the method includes adding an effective amount of a composition comprising a reaction product of a medium to high carbon count phosphate ester or a mixture medium to high carbon count phosphate esters and an amine or a mixture of amines to an aqueous or non-aqueous fluid, where the fluid is in contact with surfaces of piping, pipelines, flowlines or other downhole equipment and where the effective amount is sufficient for the reaction product to form an in-situ partial or complete coating on the surfaces.

Embodiments of this invention provide methods for inhibiting and managing corrosion in high temperature environments, where the methods include contacting surfaces of piping, pipelines, flowlines or other downhole equipment with an effective amount of a composition comprising a reaction product of a medium to high carbon count phosphate ester or a mixture medium to high carbon count phosphate esters and an amine or a mixture of amines, where the amount is sufficient for the reaction product to form a partial or complete coating on the surfaces. The methods also include flowing an aqueous or non-aqueous fluid at high temperature through the coated piping, pipelines, flowlines or other downhole equipment, where the partial or complete coating reduces corrosion of the surfaces and/or protects the surfaces from corrosion.

Embodiments of this invention provide methods for inhibiting and managing corrosion in high temperature environments, where the methods include contacting surfaces of piping, pipelines, flowlines or other downhole equipment with an effective amount of a composition comprising a reaction product of a medium to high carbon count phosphate ester or a mixture medium to high carbon count phosphate esters and an amine or a mixture of amines, where the effective amount is sufficient for the reaction product to form a partial or complete coating on the surfaces. The methods also include flowing an aqueous or non-aqueous fluid at high temperature through the coated piping, pipelines, flowlines or other downhole equipment, where the coating reduces corrosion of the surfaces and/or protects the surfaces from corrosion. The methods also include adding an additional amount of the composition, where the additional namount is sufficient to maintain the partial or complete coating on the surfaces.

### DEFINITIONS OF THE INVENTION

The term “under-balanced and/or managed pressure drilling fluid” means a drilling fluid having a hydrostatic density (pressure) lower or equal to a formation density (pressure). For example, if a known formation at 10,000 ft (True Vertical Depth—TVD) has a hydrostatic pressure of 5,000 psi or 9.6 lbm/gal, an under-balanced drilling fluid would have a hydrostatic pressure less than or equal to 9.6 lbm/gal. Most under-balanced and/or managed pressure drilling fluids include at least a density reduction additive. Other additive many include a corrosion inhibitor, a pH modifier and a shale inhibitor.

The term “ppg” means pounds per gallon (lb/gal) and is a measure of density.

The term “SG” means specific gravity.

The term “MPY” means mils per year.

The term “substantially non-corrosive” means that the phosphate brines have an MPY value of less than or equal to 350. In certain embodiments, the phosphate brines have an MPY value of less than or equal to 300. In certain embodiments, the phosphate brines have an MPY value of less than or equal to 250. In certain embodiments, the phosphate brines have an MPY value of less than or equal to 200. In certain embodiments, the phosphate brines have an MPY value of less than or equal to 175.

The term “substantially” means that the value or effect is at least 80% of being complete. In certain embodiments, the term means that the value of effect is at least 85% of being complete. In certain embodiments, the term means that the value of effect is at least 90% of being complete. In certain embodiments, the term means that the value of effect is at least 95% of being complete. In certain embodiments, the term means that the value of effect is at least 99% of being complete.

The term “about” means that the value or effect is at least 90% of being complete. In certain embodiments, the term means that the value of effect is at least 95% of being complete. In certain embodiments, the term means that the value of effect is at least 99% of being complete.

### DETAILED DESCRIPTION OF THE INVENTION

The inventors have found that corrosion inhibiting and corrosion management systems can be constructed for aqueous and non-aqueous media for use a relative low dosages, generally less than about 5,000 ppm and for use at relatively high temperature, generally up to 500° F. (260° C.), i.e., the systems are heat stable up to 500° F. (260° C.). In certain embodiment, the compositions are heat stable up to 600° F. (315.5° C.). In certain embodiment, the compositions are heat stable up to 650° F. (343.3° C.). In certain embodiment, the compositions are heat stable up to 700° F. (371.1° C.). In certain embodiment, the compositions are heat stable up to 750° F. (398.9° C.). The corrosion inhibiting and corrosion management systems are ideally well suited for use in low to moderate temperature geothermal applications and other high temperature applications, where metal surfaces are in contact with highly corrosive fluids, especially, highly corrosive aqueous fluids. In certain embodiments, the systems of this invention are heat stable in a temperature range between 100° F. (37.8° C.) and 750° F. (398.9° C.). In certain embodiments, the systems of this invention are heat stable in a temperature range between 200° F. (37.8° C.) and 700° F. (371.1° C.). In certain embodiments, the systems of this invention are heat stable in a temperature range between 300° F. (148.9° C.) and 700° F. (371.1° C.). In certain embodiments, the systems of this invention are heat stable in a temperature range between 300° F. (148.9° C.) and 650° F. (343.3° C.). In certain embodiments, the systems of this invention are heat stable in a temperature range between 300° F. (148.9° C.) and 600° F. (315.6° C.). In certain embodiments, the systems of this invention are heat stable in a temperature range between 400° F. (204.4° C.) and 700° F. (371.1° C.). In certain embodiments, the systems of this invention are heat stable in a temperature range between 400° F. (204.4° C.) and 650° F. (343.3° C.). In certain embodiments, the systems of this invention are heat stable in a temperature range between 400° F. (204.4° C.) and 600° F. (315.6° C.).

The inventors have found that the corrosion inhibiting and managements systems of this invention are also well suited for use in foamed fluids for high temperature drilling applications, where the corrosion systems may be used at relative low levels without reducing their effectiveness at temperatures up to 750° F. (398.9° C.). In certain embodiments, the temperatures are up to 700° F. (371.1° C.). In certain embodiments, the temperatures are up to 650° F. (343.3° C.). In certain embodiments, the temperatures are up to 600° F. (315.5° C.). The inhibiting systems are thought to coat metal surfaces forming a partial or complete coating that protects metal surfaces exposed to the drilling fluids especially in high temperature application. In certain embodiments, the temperature ranges between about 100° F. (37.8° C.) and about 750° F. (398.9° C.), alternatively, between about 35° C. and about 400° C. In other embodiments, the temperature ranges from about 100° C. and about 400° C. In other embodiments, the temperature ranges from about 150° C. and about 400° C. In other embodiments, the temperature ranges from about 200° C. and about 400° C. In other embodiments, the temperature ranges from about 225° C. and about 400° C. In other embodiments, the temperature ranges from about 225° C. and about 350° C. In other embodiments, the temperature ranges from about 225° C. and about 325° C. The inventors believe that the corrosion inhibiting and management systems of this invention are unique for geothermal type applications. The present corrosion inhibiting and management systems, unlike conventional corrosion inhibiting and management systems that required extremely high concentrations to provide adequate corrosion inhibition, control and management, the systems of this invention provide adequate corrosion inhibition, control and management at relatively low concentrations. The inventors have found that the corrosion inhibiting and management systems of this invention, based on their unique chemistry, are usable at lower concentrations and at higher temperatures than corrosion inhibiting and management systems currently available on the market today.

### Suitable Reagents for Use in the Invention

Suitable amines for use in the invention include, without limitation, C₈-C₁₆ carbyl diamines, C₈-C₁₆ carbyl triamines, C₅-C₈ saturated heterocyclic amines, alkylated C₅-C₈ saturated heterocyclic amines, aromatic C₈-C₁₆ heterocyclic amines, alkylated aromatic C₈-C₁₆ heterocyclic amines or mixtures and combinations thereof. Exemplary examples of C₅-C₈ saturated heterocyclic amines include, without limitation, pyrrolidine, alkylated pyrrolidine, imidazolidine, alkylated imidazolidine, pyrazolidine, alkylated pyrazolidine, oxazolidine, alkylated oxazolidine, isoxazolidine, alkylated isoxazolidine, piperidine, alkylated piperidines, piperizine, alkylated piperizines, morpholine, alkylated morpholines, azepane, alkylated azepane, azocane, alkylated azocanes and mixtures or combinations thereof. Exemplary examples of aromatic C₈-C₁₆ heterocyclic amines include, without limitation, indole, alkylated indoles, quinoline, alkylated quinolines, isoquinoline, alkylated isoquinolines and mixture or combinations thereof. The term alkylated means that the compounds may include one or more alkyl groups having from 1 to about 12 carbon atoms, where one or more of the carbon atoms may be replaced by oxygen atoms and where one or more hydrogen atoms may be replace with one or more fluorine atoms, chlorine atoms, amides, alkoxides, or other mono-valent groups that are substantially inert in the reaction or treating environments. In the case of C₈-C₁₆ carbyl diamines, the diamines may be any diamine including at least one primary amino group or secondary amino group. In the case of C₈-C₁₆ carbyl triamines, the triamines may be any triamine including at least one primary amino group or secondary amino group. Exemplary examples of amines useful in the practice of this invention include, without limitation, 2-methylquinoline (quinaldine), Amine C-6 available from Hunstman, a morpholine residue obtained from the reaction product of ethylene glycol (EG) and ammonia (NH₃), 90% pure t-butyl morpholine (TBM-90), a triamino nonane (TAN) available from Nova Molecular Technologies, Inc. of Janesville, Wis. and mixtures or combinations thereof.

Suitable phosphate esters for use in the invention include, without limitation, medium to high carbon count (medium to long chain) phosphate esters that are capable of reacting with the amines listed above to form reaction products capable of forming a partial or complete coating on surfaces exposed to highly corrosive, high temperature fluids, especially surfaces of pipes, pipelines, flow lines, downhole equipment, and/or other equipment exposed to corrosive aqueous or non-aqueous fluids at moderate to high temperatures between about 100° C. and about 400° C. Exemplary examples of such phosphate esters include, without limitation, phosphate esters of the general formulas P(O)(OR³)(OR⁴)(OR⁵), P(O)(OH)ₓ(OR⁶)_{y}, or mixture or combinations thereof, where R³, R⁴, R⁵ and R⁶ are independently C₆-C₁₄ carbyl groups having the required hydrogen atoms to satisfy valence requirements and x+y=3. The carbyl groups R³, R⁴, R⁵ and R⁶ may have one or more of their carbon atoms replaced by one or more hetero atoms selected from the group consisting of oxygen atoms and may have one or more of their hydrogen atoms replaced by one or more single valence atoms selected from the group consisting of fluorine atoms, chlorine atoms, alkyoxides, amides, or mixtures or combinations thereof. The phosphate esters are generally reaction products between alkanols and a phosphate donor such as phosphoric acid, phosphorus pentoxide, other similar phosphate donors or mixtures or combinations thereof. In certain embodiments, the R³, R⁴, R⁵ and R⁶ are independently C₆-C₁₆ carbyl groups. In other embodiments, the R³, R⁴, R⁵ and R⁶ are independently C₈-C₁₄ carbyl groups. In other embodiments, R³, R⁴, R⁵ and R⁶ are independently C₈-C₁₂ carbyl groups. In other embodiments, R³, R⁴, R⁵ and R⁶ are independently C₈-C₁₀ carbyl groups.

### EXPERIMENTS OF THE INVENTION

### Example 1

The following example illustrates the preparation of a corrosion inhibitor of this invention comprising a reaction product of a C8-C10 phosphate material, PE810, and a triaimine material, Triaminononane (TAN), CI-1. (D-314-11).

Table of Raw Materials

| Material | Amount (lb) | Composition (weight %) |
|---|---|---|
| PE810 | 0.3111 | 31.11 |
| Triaminononane (TAN) | 0.6889 | 68.89 |
| Total | 1.00 | 100.00 |

A reaction vessel was cleaned to a pristine clean condition. The indicated amount of TAN was added to vessel. Next, the indicated amount of PE810 was gradually added to the vessel over a 30 minute period of time with continuous, thorough mixing. The heat of neutralization may cause the reaction mixture to rise rapidly to a temperature between about 105° F. (40.6° C.) and 120° F. (48.9° C.). The color of the reaction mixtures changed from deep brown to light brown. The reaction mixture was continuously mixed for an additional 1 hour. The composition had the following characteristics: (1) pH neat between 9.46 and 9.54; (2) a specific gravity between 1.005 and 1.020; (3) a clear appearance, and (4) a brown color.

### Example 2

The following example illustrates the preparation of a corrosion inhibitor of this invention comprising a reaction product of a C8-C10 phosphate material, PE810, and a morpholine-containing material, Amine C-6, CI-3 (D-315-11).

Table of Raw Materials

| Material | Amount (lb) | Composition (weight %) |
|---|---|---|
| PE810 | 0.1533 | 15.33 |
| Amine C-6 | 0.8467 | 84.67 |
| Total | 1.00 | 100.00 |

A reaction vessel was cleaned to a pristine clean condition. The indicated amount of C6 Amine was added to vessel. Next, the indicated amount of PE810 was gradually added to the vessel over 30 minutes with continuous thorough mixing. The heat of neutralization may cause the reaction mixture to rise rapidly to a temperature between about 105° F. (40.6° C.) and 115° F. (46.1° C.). The color of the reaction changed from deep brown to black. The composition was continuously mixed for an additional 1 hour. The composition had the following characteristics: (1) pH neat between 9.24 and 9.32; (2) a specific gravity between 1.10 and 1.13; (3) an opaque appearance; (4) a black color; and (5) an ammonia odor.

### Examples 3-8 and Examples C1 & C2

These examples were prepared in a manner analogous to the preparations shown in Examples 1 and 2. Examples C1 and C2 are comparative examples were the phosphate ester material, PE810 was simply neutralized with either NaOH or NH₄OH. The following tables list the corrosion inhibitors and starting amine materials, respectively.

Corrosion Inhibitors (CI)

| ID | Description |
|---|---|
| CI-A | TAN-PE810 Rxn |
| CI-B | TBM-90-PE810 Rxn |
| CI-C | C₆ Amine-PE810 Rxn |
| CI-D | C₆ Amine-PE810 Rxn |
| CI-E | |
| CI-F | Quinaldine-PE810 Rxn |
| CI-G | (TAN-PE810 Rxn) + EG + Cinnamaldehyde |
| CI-H | [(Quinaldine-PE810 Rxn) + Higlyme] (41%) |
| CI-I | [(Quinaldine-PE810 Rxn) + Higlyme](67%) |
| CI-1 | PE810 + NaOH |
| CI-2 | PE810 + NH₄OH |

Starting Materials

| Reagent | Description |
|---|---|
| PE810 | C₈-C₁₀ phosphate esters |
| C6-Amine | Rxn products of EG and NH₃ - crude morpholine |
| TBM-90 | t-Butyl Morpholine, 90% pure |
| TAN | Triamino nonane |
| EG | Ethylene Glycol |

### Testing of Corrosion Inhibitors

Solutions including the corrosion inhibitors of this invention and comparative systems were tested by placing a coupon in a pressure cell. Once the coupon is in the pressure cell, an indicated volume of an inhibiting system was added to the cell. The cell was then sealed and pressurized. The pressurized cell was then placed in an oven and heated to the test temperature. In some of these corrosion tests, the corrosion inhibitors were added to a foam solution including OFHT, which is OmniFoam™ HT, a high temperature stable foamer available from Weatherford International.

Table I tabulates the results of a corrosion test performed at 450° F. and 500 psi for 5 days (120 hours).

TABLE I
Corrosion Test at 450° F., 500 psi and 300 mL for 5 Days (120 h)

| Test Solution | Wᵢ | W_{f} | Corr^{a} | Corr^{b} | Pitting | Solids |
|---|---|---|---|---|---|---|
| Fresh Water + 1.0% CI-A + 0.5% OFHT | 21.0833 | 21.0772 | 1.0 | 0.04 | None | None |
| Fresh Water + 0.5% CI-A + 0.5% OFHT | 21.3266 | 21.3198 | 1.1 | 0.05 | None | Slight |
| Fresh Water + 1.0% CI-C + 0.5% OFHT | 21.3262 | 21.3023 | 3.9 | 0.16 | Minor | None |
| Fresh Water + 0.5% CI-C + 0.5% OFHT | 21.2977 | 21.2650 | 5.3 | 0.22 | None | None |
| Fresh Water + 1.0% CI-D + 0.5% OFHT | 21.1905 | 21.1397 | 8.3 | 0.34 | None | None |
| Fresh Water + 0.5% CI-D + 0.5% OFHT + | 21.2600 | 21.2129 | 7.7 | 0.31 | None | None |
| pH 9.54 KOH | | | | | | |
| Fresh Water + 1.0% CI-F + 0.5% OFHT + | 21.3071 | 21.2301 | 12.6 | 0.51 | None | None |
| pH 9.53 KOH | | | | | | |
| Fresh Water + 0.5% CI-F + 0.5% OFHT + | 21.4218 | 21.3395 | 13.5 | 0.55 | None | Deposits |
| pH 9.71 KOH | | | | | | |

^{a}Corrosion in MPY and
^{b}Corrosion in lb/ft²/yr

The data showed that the corrosion inhibitors of this invention all had a corrosion rate measured in lb/ft²/yr of less than 1.0. In fact, most were below about 0.5. Alternatively, the corrosion inhibitors of this invention all had a corrosion rate measured in MPY of less than about 15, and most below about 10. Of interest, CI-A and CI-C showed similar corrosion inhibition at 1.0 wt. % and 0.5 wt. % concentration is the test solutions evidencing the corrosion inhibitors CI-A and CI-C are effective at relatively low concentrations.

Table II tabulates the results of a corrosion test performed at 450° F. and 500 psi for 2 days (48 hours).

TABLE II
Corrosion Test at 450° F., 500 psi and 300 mL for 2 Days (48 h)

| Test Solution | Wᵢ | W_{f} | Corr^{a} | Corr^{b} | Pitting | Solids |
|---|---|---|---|---|---|---|
| Fresh Water + 1.0% CI-A + 0.5% OFHT | 22.0305 | 22.0251 | 2.2 | 0.09 | None | Deposits |
| Fresh Water + 1.0% CI-B + 0.5% OFHT | 22.0520 | 22.0347 | 7.1 | 0.29 | None | None |
| Fresh Water + 1.0% CI-C + 0.5% OFHT | 21.8560 | 21.8287 | 11.2 | 0.45 | None | None |
| Fresh Water + 1.0% CI-D + 0.5% OFHT | 20.7912 | 20.7637 | 11.2 | 0.46 | Minor | None |
| Fresh Water + 1.0% CI-F + 0.5% OFHT | 21.0096 | 20.9408 | 28.1 | 1.14 | Minor | Deposits |
| Fresh Water + 1.0% CI-G + 0.5% OFHT | 21.2680 | 21.2572 | 4.4 | 0.18 | None | Deposits |
| Fresh Water + 1.0% CI-H + 0.5% OFHT + | 21.2466 | 21.1446 | 41.7 | 1.69 | Minor | Deposits |
| pH 9.70 KOH | | | | | | |
| Fresh Water + 0.5% CI-C + 0.5% OFHT | 21.6185 | 21.5993 | 7.8 | 0.32 | None | None |
| Fresh Wate + 0.5% CI-C + 0.5% OFHT + | 21.7103 | 21.6989 | 4.7 | 0.19 | None | None |
| 0.5% CF1 + pH 9.50 KOH | | | | | | |
| Fresh Water + 0.5% CI-D + 0.5% OFHT + | 21.6514 | 21.6259 | 10.4 | 0.42 | None | None |
| pH 9.55 KOH | | | | | | |
| Fresh Water + 0.5% CI-A + 0.5% OFHT | 22.0030 | 21.9944 | 3.5 | 0.14 | None | Deposits |
| Fresh Water + 0.5% CI-F + 0.5% OFHT | 21.8914 | 21.8748 | 6.8 | 0.28 | None | Deposits |
| Fresh Water + 0.25% CI-C + 0.5% OFHT | 21.9298 | 21.8226 | 43.8 | 1.78 | Minor^{c} | None |

^{a}Corrosion in MPY,
^{b}Corrosion in lb/ft²/yr and
^{c}minor pitting and uniform pitting where examined under a microscope

The data showed that most of the corrosion inhibitors of this invention had a corrosion rate measured in lb/ft²/yr of less than 1.0, with the exception of CI-F at 1.0 wt. %, CI-H at 1.0 wt. % at pH 9.7, and CI-C at 0.25 wt. %.

Table III tabulates the results of a corrosion test performed at 450° F. and 500 psi for 5 days (120 hours).

TABLE III
Corrosion Test at 450° F., 500 psi, and 300 mL Overnight (16 h)

| Test Solution | Wᵢ | W_{f} | Corr^{a} | Corr^{b} | Pitting | Solids |
|---|---|---|---|---|---|---|
| Fresh Water + 1.0% CI-A + 0.5% OFHT | 21.8631 | 21.8599 | 2.6 | 0.11 | None | Deposits |
| Fresh Water + 1.0% CI-B + 0.5% OFHT | 21.7970 | 21.7861 | 8.9 | 0.36 | None | None |
| Fresh Water + 1.0% CI-D + 0.5% OFHT | 21.8848 | 21.8555 | 23.9 | 0.97 | Minor | None |
| Fresh Water + 1.0% CI-C + 0.5% OFHT | 21.7396 | 21.7268 | 10.5 | 0.43 | None | None |
| Fresh Water + 1.0% CI-F + 0.5% OFHT | 21.9602 | 21.9308 | 24.0 | 0.98 | None | Deposits |
| Fresh Water + 1.0% CI-G + 0.5% OFHT | 21.9278 | 21.9193 | 6.9 | 0.28 | None | Deposits |
| Fresh Water + 1.0% CI-H + 0.5% OFHT + | 21.8857 | 21.8264 | 48.5 | 1.97 | Minor | Deposits |
| pH 9.76 KOH | | | | | | |
| Fresh Water + 0.5% CI-C + 0.5% OFHT | 22.0444 | 22.0280 | 13.4 | 0.54 | None | None |
| Fresh Water + 0.5% CI-C + 0.5% OFHT + | 22.1109 | 22.1013 | 7.8 | 0.32 | None | None |
| 0.5% CF1 + pH 9.60 KOH | | | | | | |
| Fresh Water + 0.5% CI-D + 0.5% OFHT | 22.1198 | 22.1006 | 15.7 | 0.64 | None | None |

^{a}Corrosion in MPY and
^{b}Corrosion in lb/ft²/yr

The data showed that most of the corrosion inhibitors of this invention had a corrosion rate measured in lb/ft²/yr of less than 1.0, with the exception of CI-H at 1.0 wt. % at pH 9.76.

Table IV tabulates the results of corrosion test performed at 450° F. and 500 psi for control corrosion systems currently used in the art, where the solution is a fluid used in geothermal wells. Corsaf SF is a corrosion inhibitor available from Tetra Technologies, Inc. USA; while AI600 is an acid corrosion inhibitor and corrosion inhibitors. CF1 (CorrFoam 1) and A1028 (Alpha 1028) are produced by Weatherford International, USA.

TABLE IV
Comparative Geothermal Well Corrosion Test in Compressed Air @ 450° F., 300 mL, 500 psi Post Acid CleanUp

| Test Solution | Wᵢ | W_{f} | DW | Corr^{a} | Corr^{b} | Pitting | Solids |
|---|---|---|---|---|---|---|---|
| 0.5% OFHT + 1.5% AI600 | 22.0034 | 21.5530 | 0.4504 | 368.1 | 14.96 | Heavy | None |
| 0.5% OFHT + 0.5% Corsaf SF | 22.0027 | 21.4304 | 0.5723 | 467.7 | 19.01 | Heavy | None |
| 0.5% OFHT + 1.5% AI600 + 0.5% Corsaf SF | 21.8530 | 21.3762 | 0.4768 | 389.7 | 15.84 | Heavy | Some |
| 0.5% OFHT + 1.5% AI600 + 0.5% CF1 | 21.8576 | 21.6414 | 0.2162 | 176.7 | 7.18 | Medium | None |
| 0.5% OFHT + 1.5% AI600 + 1.0% CF1 | 21.8968 | 21.7258 | 0.1710 | 139.8 | 5.68 | Medium | Some |
| 0.5% OFHT + 1.5% AI600 + 0.5% A1028 | 21.9039 | 21.4189 | 0.4850 | 396.4 | 16.11 | Heavy | Some |
| 0.5% OFHT + 2.0% CF1 + 0.5% AI600 + pH 11 KOH | 21.7446 | 21.7188 | 0.0258 | 21.1 | 0.86 | None | None |
| 0.5% OFHT + 1.0% CI-A | 21.8631 | 21.8599 | 0.0032 | 2.6 | 0.11 | None | Deposits |

^{a}Corrosion in MPY and
^{b}Corrosion in lb/ft²/yr

The data showed that most of the conventional corrosion inhibitors, and when blends of same were used, all had a corrosion rate measured in lb/ft²/yr of greater than 5.0, except for 0.5% OFHT+2.0% CF1+0.5% AI600+pH 11 KOH, and thus exemplifies the superiority of invented inhibitor CI-A under test conditions.

Table V tabulates the results of a corrosion test performed at 450° F. and 500 psi for 6 days (120 hours) run in triplicate for tap water and 5000 ppm OFHT and 5000 ppm OFHT with the indicated corrosion inhibitor CI-A, CI-C and CI-D of this invention.

TABLE V
Autoclave Corrosion Test Result - 6 Days
(144 h) at 500° F. at 2,500 to 2,800 psi

| | | | | | Average |
|---|---|---|---|---|---|
| Contents | Wᵢ (g) | W_{f} (g) | ΔW (g) | lb/ft³ | (lb/ft³) |
| Tap Water | 38.2343 | 38.2331 | 0.0012 | 0.0001 | 0.0001 |
| Tap Water | 37.7037 | 37.7029 | 0.0008 | 0.0001 | |
| Tap Water | 36.8144 | 36.8140 | 0.0004 | 0.0000 | |
| 5000 ppm OFHT | 37.3794 | 37.3785 | 0.0009 | 0.0001 | 0.0002 |
| 5000 ppm OFHT | 37.8637 | 37.8621 | 0.0016 | 0.0001 | |
| 5000 ppm OFHT | 37.2834 | 37.2789 | 0.0045 | 0.0003 | |
| 5000 ppm OFHT + CI-A | 37.9033 | 37.8936 | 0.0097 | 0.0007 | 0.0005 |
| 5000 ppm OFHT + CI-A | 37.7835 | 37.7731 | 0.0104 | 0.0007 | |
| 5000 ppm OFHT + CI-A | 41.6546 | 41.6536 | 0.0010 | 0.0001 | |
| 5000 ppm OFHT + CI-C | 38.3825 | 38.3707 | 0.0118 | 0.0008 | 0.0005 |
| 5000 ppm OFHT + CI-C | 36.1786 | 36.1702 | 0.0084 | 0.0006 | |
| 5000 ppm OFHT + CI-C | 37.0413 | 37.0389 | 0.0024 | 0.0002 | |
| (5 mL) | | | | | |
| 5000 ppm OFHT + CI-D | 37.7980 | 37.7976 | 0.0004 | 0.0000 | 0.0003 |
| 5000 ppm OFHT + CI-D | 36.6592 | 36.6540 | 0.0052 | 0.0004 | |
| 5000 ppm OFHT + CI-D | 39.8680 | 39.8608 | 0.0072 | 0.0005 | |

The data showed that the systems including the corrosion inhibitors CI-A, CI-C and CI-D are stable at 500° F. at 2,500 psi and offered acceptable performance. That is, they all had a lb/ft³ value of less than 0.04, a value accepted in the industry as a value evidencing good performance, as such, performing well over a 100 times more than acceptable level.

Table VI tabulates the results of a corrosion test performed at 450° F. and 500 psi in the presence of 20 vol. % CO₂.

TABLE VI
Corrosion Test @ 450° F., 300 mL, & 500 psi - Post Acid CleanUp

| Gas | Test Solution | Wᵢ | W_{f} | ΔW | Corr^{a} | Corr^{b} | Pitting | Solids |
|---|---|---|---|---|---|---|---|---|
| 20% CO₂ | FW^{C} + 0.5% OFHT | 21.4520 | 21.4140 | 0.0380 | 31.1 | 1.26 | Slight | Slight |
| 20% CO₂ | FW^{C} + 0.5% OFHT + 0.5% CI-A | 21.3222 | 21.3121 | 0.0101 | 8.3 | 0.34 | None | None |
| 20% CO₂ | FW^{C} + 0.5% OFHT + 0.5% CI-C | 21.4037 | 21.3961 | 0.0076 | 6.2 | 0.25 | None | None |
| 20% CO₂ | FW^{C} + 0.5% OFHT + 0.5% CI-C-90 | 21.2385 | 21.2127 | 0.0258 | 21.1 | 0.86 | None | None |
| 20% CO₂ | FW^{C} + 0.5% OFHT + 0.5% CI-03 | 21.0913 | 21.0847 | 0.0066 | 5.4 | 0.22 | None | None |
| 20% CO₂ | FW^{C} + 0.5% OFHT + 0.5% CI-04 | 21.1277 | 21.1240 | 0.0037 | 3.0 | 0.12 | None | None |
| 20% CO₂ | FW^{C} + 0.5% OFHT + 0.75% CI-A | 21.5096 | 21.5009 | 0.0087 | 7.1 | 0.29 | None | None |
| 20% CO₂ | FW^{C} + 0.5% OFHT + 1.0% TAN | 21.2013 | 21.1939 | 0.0074 | 6.0 | 0.25 | None | None |
| 20% CO₂ | FW^{C} + 0.5% OFHT + 1.2% TAN | 21.7133 | 21.7096 | 0.0037 | 3.0 | 0.12 | None | None |

^{a}Corrosion in MPY,
^{b}Corrosion in lb/ft²/yr and
^{c}Fresh Water

Test results in Table VI further exemplify desirable inhibiting performance of the invented products in a freshwater environment.

Table VII tabulates the results of a corrosion test performed at 450° F. and 500 psi in fresh water.

TABLE VII
Geothermal Corrosion Test in Helium @ 450° F., 300 mL, 500 psi Fresh Water

| Test Solution | Wᵢ | W_{f} | ΔW | Corr^{a} | Corr^{b} | Pitting | Solids |
|---|---|---|---|---|---|---|---|
| Fresh Water Blank | 21.7141 | 21.6769 | 0.0372 | 30.4 | 1.24 | Slight | None |
| Fresh Water + 0.5% CI-A + 0.5% OFHT | 21.4401 | 21.4338 | 0.0063 | 5.1 | 0.21 | None | None |
| Fresh Water + 1.0% CI-A + 0.5% OFHT | 21.6651 | 21.6590 | 0.0061 | 5.0 | 0.20 | None | None |
| Fresh Water + 0.5% CI-B + 0.5% OFHT | 21.8417 | 21.8224 | 0.0193 | 15.8 | 0.64 | None | M/T |
| Fresh Water + 0.5% CI-C + 0.5% OFHT | 22.0016 | 21.9823 | 0.0193 | 15.8 | 0.64 | None | None |
| Fresh Water + 1.0% CI-D + 0.5% OFHT | 21.9864 | 21.9677 | 0.0187 | 15.3 | 0.62 | None | None |
| Fresh Water + 0.5% CI-E + 0.5% OFHT | 21.8361 | 21.8209 | 0.0152 | 12.4 | 0.50 | None | None |
| Fresh Water + 0.5% CI-F + 0.5% OFHT | 21.5663 | 21.5595 | 0.0068 | 5.6 | 0.23 | None | None |
| Fresh Water + 0.5% CI-G + 0.5% OFHT | 21.4587 | 21.4505 | 0.0082 | 6.7 | 0.27 | None | None |
| Fresh Water + 1.0% CI-G + 0.5% OFHT | 21.7762 | 21.7671 | 0.0091 | 7.4 | 0.30 | None | M/T |

^{a}Corrosion in MPY and
^{b}Corrosion in lb/ft²/yr

The data showed that the systems including the corrosion inhibitors of this invention had all a corrosion rate measured in lb/ft²/yr of less than 1.0.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

1. A corrosion inhibiting and corrosion management system comprising:
a reaction product of:
a medium to high carbon count phosphate ester or a mixture medium to high carbon count phosphate esters and
an amine or a mixture of amines,
where the reaction product forms a partial or complete coating on surfaces of piping, flowlines or other downhole equipment in contact with an aqueous or non-aqueous fluid.

2. The system of claim 1, wherein the medium to high carbon count phosphate ester comprises a reaction product between a phosphate donor and a medium to high carbon count alcohol or mixture of medium to high carbon count alcohol.

3. The system of claim 1, wherein alcohols having between 6 and 24 carbon atoms.

4. The system of claim 1, wherein the alcohols have between 8 and 16 carbon atoms.

5. The system of claim 1, wherein the alcohols have between 8 and 12 carbon atoms.

6. The system of claim 1, wherein the amines are selected from the group consisting of C₈-C₁₆ carbyl diamines, C₈-C₁₆ carbyl triamines, C₅-C₈ saturated heterocyclic amines, alkylated C₅-C₈ saturated heterocyclic amines, aromatic C₈-C₁₆ heterocyclic amines, alkylated aromatic C₈-C₁₆ heterocyclic amines and mixtures or combinations thereof, where alkyl group or groups of the alkylated compounds independently include from 1 to about 12 carbon atoms, where one or more of the carbon atoms may be replaced by oxygen atoms and where one or more hydrogen atoms may be replace with one or more fluorine atoms, chlorine atoms, amides, alkoxides, or other mono-valent groups that are substantially inert in the reaction or treating environments.

7. The system of claim 6, wherein the C₅-C₈ saturated heterocyclic amines are selected from the group consisting of pyrrolidine, alkylated pyrrolidine, imidazolidine, alkylated imidazolidine, pyrazolidine, alkylated pyrazolidine, oxazolidine, alkylated oxazolidine, isoxazolidine, alkylated isoxazolidine, piperidine, alkylated piperidines, piperizine, alkylated piperizines, morpholine, alkylated morpholines, azepane, alkylated azepane, azocane, alkylated azocanes and mixtures or combinations thereof.

8. The system of claim 6, wherein the aromatic C₈-C₁₆ heterocyclic amines are selected from the group consisting of indole, alkylated indoles, quinoline, alkylated quinolines, isoquinoline, alkylated isoquinolines and mixture or combinations thereof.

9. The system of claim 6, wherein the C₈-C₁₆ carbyl diamines include at least one primary amino group or secondary amino group.

10. The system of claim 6, wherein the C₈-C₁₆ carbyl triamines include at least one primary amino group or secondary amino group.

11. A method for inhibiting and managing corrosion in high temperature environments comprising:
adding an effective amount of a composition comprising a reaction product of a medium to high carbon count phosphate ester or a mixture medium to high carbon count phosphate esters and an amine or a mixture of amines to an aqueous or non-aqueous fluid, where the fluid is in contact with surfaces of piping, pipelines, flowlines or other downhole equipment and where the effective amount is sufficient for the reaction product to form an in-situ partial or complete coating on the surfaces.

12. The method of claim 11, wherein the medium to high carbon count phosphate ester comprises a reaction product between a phosphate donor and a medium to high carbon count alcohol or mixture of medium to high carbon count alcohol.

13. The method of claim 11, wherein alcohols having between 6 and 24 carbon atoms.

14. The method of claim 11, wherein the alcohols have between 8 and 16 carbon atoms.

15. The method of claim 11, wherein the alcohols have between 8 and 12 carbon atoms.

16. The method of claim 11, wherein the amines are selected from the group consisting of C₈-C₁₆ carbyl diamines, C₈-C₁₆ carbyl triamines, C₅-C₈ saturated heterocyclic amines, alkylated C₅-C₈ saturated heterocyclic amines, aromatic C₈-C₁₆ heterocyclic amines, alkylated aromatic C₈-C₁₆ heterocyclic amines and mixtures or combinations thereof, where alkyl group or groups of the alkylated compounds independently include from 1 to about 12 carbon atoms, where one or more of the carbon atoms may be replaced by oxygen atoms and where one or more hydrogen atoms may be replace with one or more fluorine atoms, chlorine atoms, amides, alkoxides, or other mono-valent groups that are substantially inert in the reaction or treating environments.

17. The method of claim 16, wherein the C₅-C₈ saturated heterocyclic amines are selected from the group consisting of pyrrolidine, alkylated pyrrolidine, imidazolidine, alkylated imidazolidine, pyrazolidine, alkylated pyrazolidine, oxazolidine, alkylated oxazolidine, isoxazolidine, alkylated isoxazolidine, piperidine, alkylated piperidines, piperizine, alkylated piperizines, morpholine, alkylated morpholines, azepane, alkylated azepane, azocane, alkylated azocanes and mixtures or combinations thereof.

18. The method of claim 16, wherein the aromatic C₈-C₁₆ heterocyclic amines are selected from the group consisting of indole, alkylated indoles, quinoline, alkylated quinolines, isoquinoline, alkylated isoquinolines and mixture or combinations thereof.

19. The method of claim 16, wherein the C₈-C₁₆ carbyl diamines include at least one primary amino group or secondary amino group.

20. The method of claim 16, wherein the C₈-C₁₆ carbyl triamines include at least one primary amino group or secondary amino group.
